(19) United States
(12) Plant Patent Application Publication
(10) Pub. No.: US 2008/0184417 P1
(43) Pub. Date: Jul. 31, 2008

(54) Lantana plant named 'Bante Reeda'

(76) Inventor: Shifeng Pan, Hollister, CA (US)

Correspondence Address: JONDLE & ASSOCIATES P.C., 858 HAPPY CANYON ROAD SUITE 230, CASTLE ROCK, CO, 80108, US

(21) Appl. No.: 11/654,927

(22) Filed: Jan. 17, 2007

(51) Int. Cl. A01H 5/00

(52) U.S. Cl. PLT/227

(57) ABSTRACT

A lantana plant particularly distinguished by deep red flowers, a continuous, abundant and mid-season flowering response, dark green foliage with ovate leaves, vigorous, compact growth, low mounding and good branching ability, is disclosed.

### GENUS AND SPECIES

Lantana camara

### VARIETY DENOMINATION

‘Bante Reeda’

### BACKGROUND OF THE NEW PLANT

The present invention comprises a new and distinct cultivar of lantana, botanically known as Lantana camara and hereinafter referred to by the cultivar name ‘Bante Reeda’. The new cultivar originated from a pollination made in July 2003 in Gilroy, Calif. The new cultivar was created from the crossing of ‘22-1’ , a fuchsia female (unpatented) proprietary line with ‘F13-1’, a red male (unpatented) proprietary line.

The new cultivar was created in July of 2003 in Gilroy, Calif. and has been asexually reproduced repeatedly by vegetative cuttings and tissue culture in Gilroy, Calif and Andijk, The Netherlands over a two and one-half-year period. The plant has also been trialed at Gilroy, Calif., Andijk, The Netherlands and Hillscheid, Germany. The present invention has been found to retain its distinctive characteristics through successive asexual propagations.

Plant Breeder's Rights for this cultivar were applied for in Germany on Nov. 27, 2006.

### SUMMARY OF THE INVENTION

The following are the most outstanding and distinguishing characteristics of the new cultivar when grown under normal horticultural practices in Gilroy, Calif.

1. Deep red flowers;

2. A continuous, abundant and mid-season flowering response;

3. Dark green foliage with ovate leaves;

4. Vigorous growth;

5. A compact and low mounding plant habit; and

6. Good branching ability.

### DESCRIPTION OF THE PHOTOGRAPHS

This new lantana plant is illustrated by the accompanying photographs which show overall plant habit including blooms, buds and foliage of the plant; the colors shown are as true as can be reasonably obtained by conventional photographic procedures. The photos are of 15-16 week-old plants in September of 2006.

FIG. 1 shows the overall plant habit, including inflorescences, buds and foliage of the whole plant.

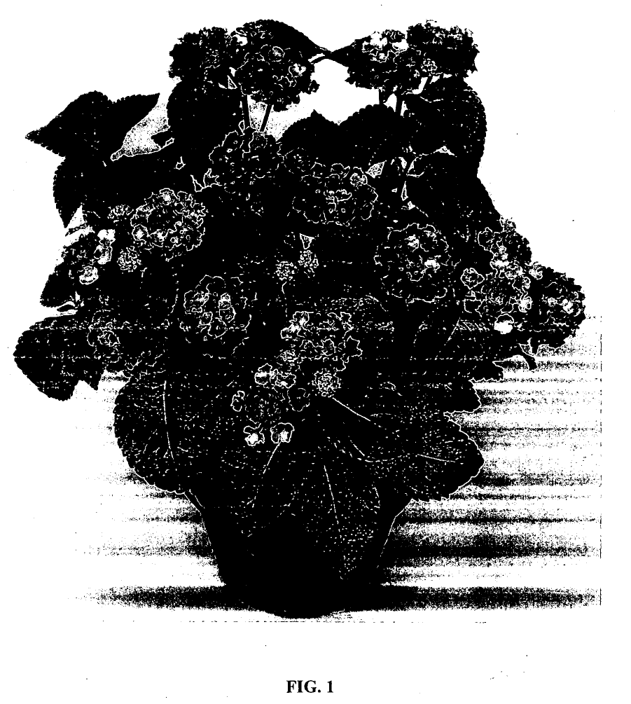

FIG. 2 shows a close-up of the mature inflorescence.

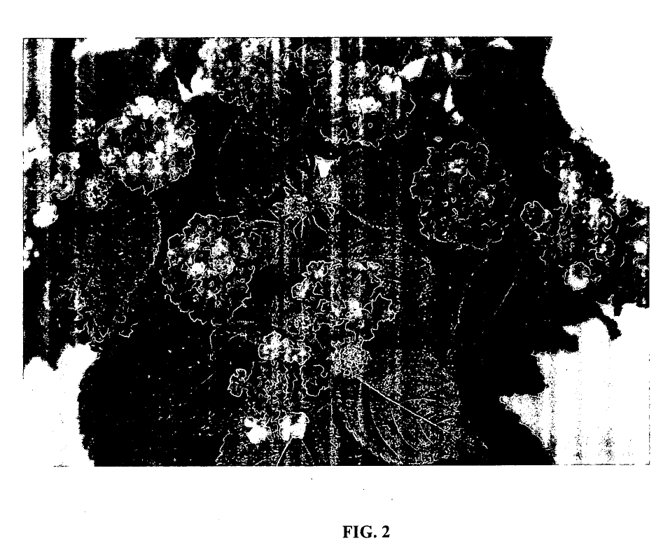

### DESCRIPTION OF THE NEW CULTIVAR

The following detailed descriptions set forth the distinctive characteristics of ‘Bante Reeda’. The data which define these characteristics were collected from asexual reproductions carried out in Gilroy, Calif. and Hillscheid, Germany. The plant history was taken on 26-week-old plants, grown in Gilroy, Calif. in 4′′ pots. Observations were made in summer and early fall. The plants had one plant growth regulator treatment and one terminal pinch after cuttings were transplanted to the 4′ pots. Color readings were taken under natural light. Color references are primarily to the R.H.S. Colour Chart of The Royal Horticultural Society of London (R.H.S.) (2001 edition).

### DETAILED BOTANICAL DESCRIPTION

Classification:
   Family.—Verbenaceae.
   Botanical.—Lantana camara.
   Common name.—Lantana.
Parentage:
   Female parent.—‘22-1’, a fuschia proprietary line (unpatented).
   Male parent.—‘F13-1’, a red proprietary line (unpatented).
Growth:
   Form.—Semi-woody, herbaceous; grown as an annual or perennial.
   Growth and branching habit.—Compact, low mounding, good branching, pinching of the apices enhances the number of branches and results in a tighter plant habit.
   Height (measured from the top of the soil).—20.0 cm to 25.0 cm.
   Width (horizontal plant diameter).—25.0 cm to 30.0 cm.
   Time to produce a finished flowering plant.—12 to 13 weeks for a 12-cm pot in the spring.
   Outdoor plant performance.—Use in containers and hanging baskets.
   Time to initiate and develop roots.—Approximately 24 days.
   Root description.—Fibrous, fleshy, white, self-branching.
Stems:
   Number of branches per plant.—6-8.
   Length.—20.0 cm to 25.0 cm.
   Diameter (measured from the midpoint).0.3 cm to 0.4 cm.
   Internode length.—1.5 cm to 3.0 cm.
   Color.— Immature (growing) stems: RHS 144B Mature (woody) stems: RHS 144B with RHS 199D and RHS N200A in irregular vertical patterns.
   Anthocyanin.—Absent.
   Texture.—Glandular hairs; scabrous, some with a black ring around their base.
Leaves:
   Arrangement.—Opposite.
   Fragrance.—Slightly sweet and spicey.
   Immature leaf.—Color: Upper surface: RHS 137A Lower surface: RHS 137C.
   Mature leaf.— Color: Upper surface.—RHS 147A, but darker Lower surface: RHS 146B Length: 4.5 cm to 6.0 cm Width: 3.5 cm to 3.9 cm Shape: Ovate Apex: Acute Base: Attenuate Margin: Serrate Texture: Rugose; glandular hairs; scabrous, bristles in the mid-vein have a black ring around the base Venation: Pinnate Venation color: RHS 144A for the lower surface, the upper surface is indistinct.
   Petioles.— Length: 1.5 cm to 2.0 cm Diameter: 0.2 cm Color: RHS 144A Texture: Glandular hairs; scabrous, some bristles with black rings around the base.
Flower buds:
   Shape.—Tubular.
   Length.—0.7 cm to 0.8 cm.
   Diameter (at the tip).—0.5 cm.
   Color (at tight bud).—RHS 179A.
Inflorescence:
   Blooming habit.—Flowers continuously from Spring through Fall; Inflorescences usually appear in pairs from a nodium with one inflorescence per leaf axil.
   Fragrance.—Sweet and slightly spicey.
   Inflorescence type.—Umbel-like flattened semi-sphere, the florets are sessile on an ovate receptacle.
   Horizontal diameter of inflorescence.—4.0 to 4.5 cm.
   Vertical depth of inflorescence.—2.0 cm to 2.5 cm.
   Number of inflorescences per plant.—20-30.
   Number of florets per inflorescence.—approximately 26.
   Peduncle.— Length: 1.0 cm to 1.5 cm Diameter: 0.2 cm to 0.25 cm Color: RHS 144A Texture: Glandular hairs; scabrous.
   Pedicels.—None, flowers are sessile.
Flowers:
   Floret form.—Salver-shaped to shallow cup-shaped and zygomorphic, with a relatively long tube at the base. Each flower is subtended by a single bract.
   Corolla diameter.—1.0 cm to 1.2 cm.
   Floret depth.—1.3 cm to 1.4 cm.
   General color.—Mostly yellow in the very opening stages, then turning through varying hues of oranges, with red mature florets. Color intensities can vary with cultural growing conditions.
   Immature flower (face).— Color: Upper surface: RHS 12A; later overlaid with RHS 32B mainly around the margin. Lower surface: RHS 24C to RHS 24D.
   Mature flower (face).— Color: Upper surface: RHS 44A around the corolla opening, RHS 32A to RHS 32B in varying intensities around the margins. Lower surface: RHS 16C to RHS 16D Corolla tube: Color: Inner surface: RHS 24C to RHS 24D Outer surface: RHS 15A Texture: Densely pubescent, most with a deep orange-red hue Length: 1.1 cm to 1.2 cm Diameter (at opening): 0.2 cm.
   Petals.— Length of lobe: 0.5 cm to 0.7 cm Diameter of lobe: 0.5 cm to 0.6 cm Lobe shape: Obovate Apex: Rounded to slightly retuse Base: Fused Margin: Entire, slightly ruffled Degree of lobation: Moderate Texture: Papillose, with a few hairs showing basally at the corolla opening.
   Bract.— Color: RHS 143C to RHS 143D with an RHS 144A apex Length: 0.4 cm to 0.5 cm Diameter: 0.2 cm Shape: Lanceolate Apex: Acute Margin: Entire Texture: Densely pubescent.
   Calyx.— General: Composed of 5 fused sepals forming a short tube around the base of each floret Shape: Tube-shaped with two short lobes Length: 0.15 cm to 0.2 cm Diameter: 0.2 cm Color: Upper surface: RHS 43B; RHS 43A but darker at the apex. Lower surface: Not observed Apex (of calyx lobe): Broadly rounded Base (of sepals): Fused Margin (of sepals): Side-margins are fused, but tips are shallowly fringed. Texture: Densely pubescent.
Reproductive organs:
   Stamens.— Quantity: 4 Filament color: RHS 9C Filament diameter: 0.3 cm to 0.5 cm Filament length: 0.05 cm Anther color: RHS 5C Anther length: 0.075 cm Anther shape: 2-lobed, oblong Pollen amount: Sparse Pollen color: RHS 18A.
   Pistil.— Quantity: One Length: 0.5 cm Stigma color: RHS 145A Style color: RHS 3C Style length: 0.3 cm.
Fruit/seed set: A very small quantity of seed is produced per plant.
Disease and insect resistance: None observed.

### COMPARISON WITH PARENTAL AND COMMERCIAL CULTIVARS

‘Bante Reeda’ differs from the female parent ‘22-1’ in that ‘Bante Reeda’ has a deep red flower color, and the female parent has a fuchsia flower color. ‘Bante Reeda’ flowers earlier and has darker green, smaller foliage than the female parent.

‘Bante Reeda’ differs from the male parent ‘F13-1’ in that ‘Bante Reeda’ is more compact and has more basal branching than the male parent. Additionally, ‘Bante Reeda’ has a deeper flower color than the male parent.

‘Bante Reeda’ differs from the commercial variety ‘Dallas Red’ (unpatented) in that ‘Bante Reeda’ has more branching, shorter internodes, a more compact habit, and slightly darker and glossier leaves. Additionally, ‘Bante Reeda’ mature flowers are a darker red and the immature flowers are a little lighter yellow than ‘Dallas Red’.

I claim:

1. A new and distinct cultivar of lantana plant as shown and described herein.
